(19) United States
(12) Patent Application Publication
(10) Pub. No.: US 2009/0054274 A1
(43) Pub. Date: Feb. 26, 2009

(54) Formation of Heteroatom Containing Derivatives of Paraffins

(76) Inventors: Howard Lam-Ho Fong, Sugar Land, TX (US); Thomas Howard Johnson, Houston, TX (US); Thomas Carl Semple, Friendswood, TX (US)

Correspondence Address: SHELL OIL COMPANY, P O BOX 2463, HOUSTON, TX, 772522463, US

(21) Appl. No.: 11/887,169

(22) PCT Filed: Mar. 24, 2006
(86) PCT No.: PCT/US06/10768

§ 371 Date: Sep. 26, 2007

Related U.S. Application Data
(60) Provisional application No. 60/665,714, filed on Mar. 28, 2005.

(51) Int. Cl. E21B 43/25; C07C 17/23; C07C 43/10

(52) U.S. Cl. 507/266; 570/261; 568/619

(57) ABSTRACT

A process by which alkyl halides may be reacted (coupled) with nucleophilic materials in the presence of a homogeneous catalyst system. The process comprises reacting (coupling) alkyl halides with a nucleophilic material in the presence of a homogeneous catalyst system to produce derivatives of alkyl halides, wherein the homogeneous catalyst system comprises at least one metal or metal compound which has the ability to form metal-halogen bonds.

### FIELD OF THE INVENTION

This invention relates to the reaction of alkyl halides with nucleophilic materials in the presence of a homogeneous catalyst. More particularly, this invention relates to paraffin coupling reactions via halogens to form heteroatom containing derivatives.

### BACKGROUND OF THE INVENTION

Alkyl alcohol alkoxylates, including secondary alkyl alcohol ethoxylates, are useful products for making detergent products and for other uses. Alkyl alcohol ethoxylates have been made by several different processes in the past.

There are commercial processes for making primary alcohols, alkoxylates, olefins, amines, sulfides, etc. directly from alkanes. These processes are all expensive and are only used to make products for use in special applications when a high price is not a deterrent in the marketplace. One such process is the Pacol Olex process wherein paraffins are first dehydrogenated to form internal olefins which are then hydroformylated to produce primary alcohols.

One process involves reaction of an internal olefin with a glycol such as diethylene glycol (DEG) and an acid catalyst such as a zeolite. Under acid conditions, the olefin will polymerize and the DEG will dehydrate. These competing reactions decrease the yield.

Another process involves the reaction of an internal halide with DEG. Very poor yields have been reported for this reaction.

Another process, which is used commercially, involves reaction of an internal alcohol with ethylene oxide and an acid catalyst to make secondary alcohol ethoxylates. This process has the disadvantage that only half of the secondary alcohol reacts with the ethylene oxide (the rest is free alcohol which is undesirable for laundry applications because of its smell). The conversion of the secondary alcohols to secondary alcohol alkoxylates is an expensive step because a separation step is needed to separate the secondary alcohol ethoxylate product, usually 2-3 mole secondary alcohol ethoxylates (SAE), from the starting material (the secondary alcohol). This thermal separation is difficult and costly. The 2-3 mole SAE can then be reacted with EO and potassium hydroxide to form 5-50 mole SAE's. It would be advantageous to find a way to make the SAE's without having to perform this separation step. The present invention provides such a process and also provides a process by which alkyl halides may be converted into a wide variety of useful products by reacting them with a wide variety of nucleophilic materials.

### SUMMARY OF THE INVENTION

This invention provides a process for paraffin coupling reactions via halogens to form heteroatom containing derivatives in which paraffins may be halogenated to form alkyl halides and alkyl halides may be reacted (coupled) with nucleophilic materials in the presence of a homogeneous catalyst system. If the product to be made is an alkyl alcohol alkoxylate, the process may be a direct alkoxylation coupling reaction because the alkyl alcohol alkoxylates are made without going through an intermediate. Carbon numbers of particular interest for making allyl alcohol alkoxylates are C₄ to C₂₀, C₆ to C₁₄, C₁₃ to C₁₇ and C₁₀ to C₁₃.

The process may comprise reacting alkyl halides with a nucleophilic material in the presence of a homogeneous catalyst system to produce derivatives of alkyl halides, wherein the homogeneous catalyst system comprises at least one metal or metal compound which has the ability to form metal-halogen bonds. In another embodiment, the process may comprise halogenating paraffins to produce alkyl halides and reacting the alkyl halides with a nucleophilic material in the presence of a homogeneous catalyst system to produce derivatives of alkyl halides, wherein the homogeneous catalyst system comprises at least one metal or metal compound which has the ability to form metal-halogen bonds.

Other embodiments include methods for enhanced oil recovery, making detergents, and making personal care compositions from paraffins and/or alkyl halides.

### DETAILED DESCRIPTION OF THE INVENTION

The present invention provides a process to convert alkyl halides directly to valuable products by reaction with nucleophilic materials in the presence of a homogeneous catalyst system. The reactions are paraffin coupling reactions via halogens to form heteroatom containing derivatives. High conversion of alkyl halides to useful products is achieved in the coupling reaction and also the selectivity of this reaction to produce the desired products is high. The use of the homogeneous catalyst system increases the reaction rate. This invention is especially advantageous in the case of making alkyl alcohol alkoxylates because it eliminates the expensive step of converting alcohols to alkyl alcohol alkoxylates.

A nucleophilic material is one that will participate in a nucleophilic reaction wherein 1) a bond is broken, i.e., a carbon-halogen bond, 2) the carbon to which the leaving group i.e., a halogen, is attached is an alkyl carbon, and 3) a bond is formed between the carbon and the nucleophilic portion, i.e., the alcohol part of, for example, diethylene glycol, of the nucleophilic material. Preferred nucleophilic materials include those which contain oxygen, nitrogen, and/or sulfur, most preferably oxygen. Dimethylaminopropylamine will react with primary alkyl halides to produce gasoline additives to clean engines. CH₃SH (MeSH) will react with primary alkyl halides to make R—S-Me which is used to control chemical reactions. Other preferred materials include glycols, thioalcohols, and alcohol amines. The most highly preferred nucleophilic materials are polyethylene glycols (PEG), polypropylene glycols, diethylene glycol (DEG), triethylene glycol (TEG), dimethylaminopropylamine, CH₃SH, monopropylene glycol (MPG) and monoethylene glycol (MEG). PEG 400 (400 molecular weight) is preferred when the alkyl alcohol alkoxylate is to be used in industrial cleaners. DEG is most preferred for making alkoxylates because of its low cost, its stability under these conditions, its ability to solubilize the catalyst, and because its boiling point helps in the downstream separation steps. DEG is the preferred nucleophilic material when the intended use is in shampoos.

In one embodiment of this invention, the nucleophilic material may serve as the medium for the homogeneous catalyst and for the reaction to take place. Glycols are preferred for making alkoxylates since they will easily solubilize the reactants and the catalyst.

In another embodiment of this invention, specific mixtures of products may be produced by selecting a desired mixture of nucleophilic materials. For example, if the desired product is a 70-30 mole % mixture of the alkoxylates of DEG and TEG, then the feed should comprise a 70-30 mole % mixture of DEG and TEG.

Direct alkoxylation coupling (DAC) represents one embodiment of the present invention. This reaction allows the direct alkoxylation of alkyl halides to form alkyl alcohol alkoxylates (AAA). The alkyl halide is reacted with a nucleophilic material in the presence of a homogeneous catalyst system to produce AAA or a mixture of AAA and olefins, wherein the homogeneous catalyst system comprises at least one metal or metal compound which has the ability to form metal-halogen bonds. The reaction may be carried out at a temperature from 100 to 200° C., preferably 140 to 160° C. In a preferred embodiment, the reactants and the catalyst are dissolved in the nucleophilic material.

The homogeneous catalyst system comprises at least one metal or metal compound which has the ability to form metal-halogen bonds. Most metals will perform this function. The purpose of the metal and metal compound is to catalyze the coupling reaction and make it go fast enough to make the process practical. Preferably, the metal is selected from metals of Groups VIII, IB and IIB of the periodic table of the elements, CAS version. The metals of Groups VIII, IB and IIB of the periodic table of the elements are also described in “Advanced Inorganic Chemistry, Fourth Edition”, Authored by F. A. Cotton and G. Wilkinson, A Wiley Interscience Publication, 1980. Particularly preferred catalysts include FeBr₃, CuBr₂, CoBr₂, MgBr₂ and ZnBr₂. Zn is most highly preferred because it gives the fastest rates and the highest yields and Br₂ is preferred because the metal-Br bond is one of the strongest metal-halogen bonds. Other metal compounds that can be added include metal acetates, carbonates, alkoxylates, nitrates, etc. because they will form metal-halogen bonds.

In one embodiment of the invention, the nucleophilic material and/or any hydrogen halide present in the coupling reaction mixture may be separated from the products. In another embodiment, any nucleophilic material which is recovered may be recycled to the coupling reaction. In another embodiment, any hydrogen halide recovered may be used for a variety of purposes including making halogen which may be used to make alkyl halides for use in this process.

In an embodiment of the invention wherein the alkyl halides are alkoxylated, alkyl alcohol alkoxylate and olefin products may be separated by first sparging the reaction mixture with an inert gas, preferably nitrogen. The rest of the separation may be carried out by phase separation. The mixture may be cooled and a solvent such as hexane or some other light hydrocarbon may be added. One advantage of the preferred nucleophilic materials of this invention which may be used for alkoxylation, polyethylene glycols, polypropylene glycols, DEG, TEG, MPG and MEG, is that they will cause the formation of the desired liquid phases without additional materials or process steps. If necessary, a phase inducing agent such as a salt (aqueous salt solution) can be used to form the desired phases.

The top layer may contain the product alkyl alcohol alkoxylates and olefins, solvent and any remaining hydrogen halide. The bottom layer may contain the nucleophilic material and the catalyst. These may be recycled to reactor.

The alkyl halides for use herein may be made by any process suitable for making alkyl halides. One method is by halogenation of alkanes as described in copending U.S. application Ser. No. 60/563,966, filed Apr. 21, 2004, entitled “Process to Convert Linear Paraffins into Alpha Olefins”, the entire disclosure of which is herein incorporated by reference, published on Nov. 3, 2005 as U.S. published patent application 2005/0245777. Another method of making alkyl halides is the Wohl-Ziegler bromination of hydrocarbons with N-bromosuccinimide. The alkyl halides for use in the coupling reaction may include mono- and dialkyl halides as well as alkyl halides containing more than 2 halogens.

Alkanes of particular interest are linear alkanes, branched alkanes, cycloalkanes, or combinations of linear alkanes and/or branched alkanes and/or cycloalkanes may be converted via halogenation to alkyl halides.

Halogenation may preferably be carried out thermally or catalytically (for example in a conventional reactor, in a catalytic distillation column, etc.), and with or without the use of a catalyst support intended to promote shape selectivity. Halogenation processes that preferentially produce monoalkyl halides (e.g., catalytic halogenation at lower temperatures, thermal halogenation at higher temperatures, etc.) may be used. One such process is the catalytic distillation process disclosed and claimed in copending, commonly assigned application entitled “CATALYTIC DISTILLATION PROCESS FOR PRIMARY HALOGENATED ALKANES”, filed concurrently herewith, which is herein incorporated by reference in its entirety. Preferred halogens are chlorine, bromine, and iodine. Particularly preferred is bromine because it is easier to regenerate than the others and it will produce more of the desired internal alkyl halides. The halogenation reaction of alkanes inherently produces a predominant amount of internal alkyl halides which are desired for the production of secondary alkyl alcohol alkoxylates.

Thermal halogenation may be carried out by introducing the halogen and the alkane to a reactor and heating the reactants to a temperature which may range from 60° C., below which the reaction rate is slow, to 200° C., which is high enough to start losing hydrogen halide. However, temperatures up to 400° C. may be used. The preferred range is from 100° C. to 150° C.

As stated above, catalytic halogenation may be carried out at lower temperature, such as from 25° C. to 400° C. The preferred temperature ranges are the same as those for thermal halogenation. Catalysts which may be used include compounds and/or complexes containing Ti, Zr, Hf, V, Nb, Ta, Cr, Mo, W, Mn, Re, Fe, Ru, Co, Rh, Ir, Ni, Pd, Pt, Cu, Ag, Au, Zn, Cd, B Al, Ga, In, Tl, Si, Ge, Sn, Pb, P, Sb, Bi, S, Cl, Br, F, Sc, Y, Mg, Ca, Sr, Ba, Na, Li, K, O, La, Ce, Pr, Nd, Sm, Eu, Gd, Th, Er, Yb, Lu and Cs or mixtures thereof. The amount of catalyst used will vary with the specific catalyst used and the reaction conditions selected but will range from 0.00001 grams to 100 grams of catalyst per gram of alkane passed over the catalyst per hour. The reaction may also be conducted in the presence of a diluent such as nitrogen, helium or argon. The process may be conducted at pressures ranging from 0.1 atm to 100 atm pressure.

Another embodiment of this invention comprises a method for enhanced oil recovery which comprises (a) making alkyl alcohol alkoxylates as described above; (b) providing the alkyl alcohol alkoxylates to at least a portion of a hydrocarbon containing formation; and (c) allowing the alkyl alcohol alkoxylates to interact with hydrocarbons in the hydrocarbon containing formation.

Hydrocarbons may be recovered from hydrocarbon containing formations by penetrating the formation with one or more wells. Hydrocarbons may flow to the surface through the wells. Conditions (e.g., permeability, hydrocarbon concentration, porosity, temperature, pressure) of the hydrocarbon containing formation may affect the economic viability of hydrocarbon production from the hydrocarbon containing formation. A hydrocarbon containing formation may have natural energy (e.g., gas, water) to aid in mobilizing hydrocarbons to the surface of the hydrocarbon containing formation. Natural energy may be in the form of water. Water may exert pressure to mobilize hydrocarbons to one or more production wells. Gas may be present in the hydrocarbon containing formation at sufficient pressures to mobilize hydrocarbons to one or more production wells. The natural energy source may become depleted over time. Supplemental recovery processes may be used to continue recovery of hydrocarbons from the hydrocarbon containing formation. Examples of supplemental processes include waterflooding, polymer flooding, alkali flooding, thermal processes, solution flooding or combinations thereof.

In an embodiment, hydrocarbons may be produced from a hydrocarbon containing formation by a method that includes treating at least a portion of the hydrocarbon containing formation with a hydrocarbon recovery composition. In certain embodiments, at least a portion of the hydrocarbon containing formation may be oil wet. In some embodiments, at least a portion of the hydrocarbon formation may include low salinity water. In other embodiments, at least a portion of the hydrocarbon containing formation may exhibit an average temperature of less than 50° C. Fluids, substances or combinations thereof may be added to at least a portion of the hydrocarbon containing formation to aid in mobilizing hydrocarbons to one or more production wells in certain embodiments. One example of such a process is described in U.S. Patent Application Publication No. 2004/0177958, which is herein incorporated by reference in its entirety.

Another embodiment comprises a method for making a detergent composition which comprises (a) making alkyl alcohol alkoxylates as described above; and (b) adding to the alkyl alcohol alkoxylates (1) at least one builder, optionally, (2) at least one co-surfactant, and, optionally, (3) other conventional detergent ingredients. Such compositions, conventional ingredients and methods for making them are described in U.S. Patent Application Publication No. 2005/0153869, which is herein incorporated by reference in its entirety.

Suitable silicate builders include water-soluble and hydrous solid types and including those having chain-, layer-, or three-dimensional-structure as well as amorphous-solid silicates or other types, for example especially adapted for use in non-structured-liquid detergents. Also suitable for use herein are synthesized crystalline ion exchange materials or hydrates thereof having chain structure and a composition represented by the following general formula in an anhydride form: xM₂O.ySiO₂.zM′O wherein M is Na and/or K, M′ is Ca and/or Mg; y/x is 0.5 to 2.0 and z/x is 0.005 to 1.0 as taught in U.S. Pat. No. 5,427,711, Sakaguchi et al, Jun. 27, 1995, incorporated herein by reference. Aluminosilicate builders, such as zeolites, are especially useful in granular detergents, but can also be incorporated in liquids, pastes or gels.

The detergent compositions according to the present invention preferably further comprise surfactants, herein also referred to as co-surfactants. It is to be understood that surfactants prepared in the manner of the present invention may be used singly in cleaning compositions or in combination with other detersive surfactants. Typically, fully formulated cleaning compositions will contain a mixture of surfactant types in order to obtain broad-scale cleaning performance over a variety of soils and stains and under a variety of usage conditions. A typical listing of anionic, nonionic, cationic, ampholytic and zwitterionic classes, and species of these co-surfactants, is given in U.S. Pat. No. 3,664,961 issued to Norris on May 23, 1972, incorporated herein by reference. Amphoteric surfactants are also described in detail in “Amphoteric Surfactants, Second Edition”, E. G. Lomax, Editor (published 1996, by Marcel Dekker, Inc.) McCutcheon's, Emulsifiers and Detergents, Annually published by M. C. Publishing Co., and Surface Active Agents and Detergents” (Vol. I and II by Schwartz, Perry and Berch), all of which are incorporated herein by reference.

Another embodiment comprises a method for making a personal care composition which comprises (a) making alkyl alcohol alkoxylates as described above; and (b) adding to the alkyl alcohol alkoxylates (1) a cosmetically acceptable vehicle and, optionally, (2) at least one sunscreen. Methods for making such compositions are described in U.S. Patent Application Publications Nos. 2005/0048091 and 2005/0196362, which are herein incorporated by reference in their entirety.

The cosmetically-acceptable vehicle is generally present in a safe and effective amount, preferably from 1% to 99.99%, more preferably from about 20% to about 99%, especially from about 60% to about 90%. The cosmetically-acceptable vehicle can contain a variety of components suitable for rendering such compositions cosmetically, aesthetically or otherwise, acceptable or to provide them with additional usage benefits. The components of the cosmetically-acceptable vehicle should be physically and chemically compatible with the branched ester component and should not unduly impair the stability, efficacy or other benefits associated with the personal care compositions of the invention.

Suitable ingredients for inclusion in the cosmetically-acceptable vehicle are well known to those skilled in the art. These include, but are not limited to, emollients, oil absorbents, antimicrobial agents, binders, buffering agents, denaturants, cosmetic astringents, film formers, humectants, surfactants, emulsifiers, sunscreen agents, oils such as vegetable oils, mineral oil and silicone oils, opacifying agents, perfumes, coloring agents, pigments, skin soothing and healing agents, preservatives, propellants, skin penetration enhancers, solvents, suspending agents, emulsifiers, cleansing agents, thickening agents, solubilizing agents, waxes, inorganic sunblocks, sunless tanning agents, antioxidants and/or free radical scavengers, chelating agents, suspending agents, sunless tanning agents, antioxidants and/or radical scavengers, anti-acne agents, anti-dandruff agents, anti-inflammatory agents, exfolients/desquamation agents, organic hydroxy acids, vitamins, natural extracts, inorganic particulates such as silica and boron nitride, deodorants and antiperspirants.

The one or more sunscreens for use herein may be selected from organic sunscreens, inorganic sunscreens and mixtures thereof. Any inorganic or organic sunscreen suitable for use in a personal care composition may be used herein. The level of sunscreen used depends on the required level of Sun Protection Factor, “SPF”. In order to provide a high level of protection from the sun, the SPF of the personal care composition should be at least 15, more preferably at least 20. Suitable inorganic sunscreens for use herein include, but are not necessarily limited to, cerium oxides, chromium oxides, cobalt oxides, iron oxides, titanium dioxide, zinc oxide and zirconium oxide and mixtures thereof. The inorganic sunscreens used herein may or may not be hydrophobically-modified, for example, silicone-treated. In preferred embodiments herein, the inorganic sunscreens are not hydrophobically-modified.

Although the examples focus on one embodiment of the invention, it is understood that the invention is not limited to such embodiment or to the embodiments disclosed, but is capable of numerous rearrangements, modifications, and substitutions of parts and elements without departing from the spirit of the invention.

### EXAMPLES

### Example 1

### Separation of Hexane, Bromohexane and Dibromohexane

A mixture of 5 grams of hexane, 5 grams of 1-Bromohexane and 5 grams of 1,2 dibromo hexane were mixed and placed in a 50 ml round bottom flask. A 200 mm Vigreux distilling column and a short path distillation column were attached to the top of the round bottom flask and heat was applied to the round bottom flask via a heating mantle. When the mixture reached 70° C., the hexane was distilled from the mixture, condensed and collected in the receiving flask. After 5 grams had been collected, no more material was condensing. The round bottom flask was heated to 160° C. and the 1-Bromohexane started to distill. 5 grams of material were collect in the receiving flask. Finally, the material remaining in the round bottom flask was tested by gas chromatography (GC) and was shown to be 1,2-dibromo hexane.

### Example 2

### Conversion of Dibromohexane to Monobromohexane

To demonstrate the conversion of a dibromohexane to a monobromohexane, 2 grams of 2,3-dibromohexane, 0.1 grams of nickel acetate and 100 mls of cyclohexane was added to a small bolt head autoclave. The autoclave was flushed 3 times with 50 psi of nitrogen and then charged with 55 psi of Hydrogen. The vessel was allowed to sit at 25° C. for two hours to activate the nickel. After two hours, a sample was taken from the autoclave via a dip tube and showed only 2,3-dibromohexane. The autoclave was heated to 180° C. and allowed to react for 60 minutes. A sample was taken and showed that half of the starting material had been converted to a mixture of the 2 and 3 bromohexane isomers with only about 1% being converted all the way to hexane.

### Example 3

### Conversion of 2-BromoOctane and 2″-Hydroxy-2′Ethoxy-2 Ethanol to 2′″-Hydroxy-2″ethoxy-2′ethoxy-2-octane

To a three neck round bottom flask was charged 200 grams of DEG or a DEG/TEG mixture, 4 grams of an alkyl halide and 1.5 grams of catalyst (see Table 1). This is the DAC process. In some experiments sodium citrate was added (see Table 1). In one neck was placed a stirring rod, in the second neck was placed a thermal well and in the last neck was placed a reflux condenser with a nitrogen flow across the top of the condenser leading to a bubbler. Heat was applied to the round bottom flask via a heating mantle. The mixture was heated to 180° C. for 10, 30 or 60 minutes (see Table 1). The reaction was cooled down to room temperature and 100 mls of n-hexane was added. To this solution, 400 mls of saturated NaCl solution was added. Mixture was transferred into a separator funnel and was extracted 3 more times with 100 mls n-hexane. Hexane was dried with MgSO₄ and filtered. The water from the aqueous solution was removed under vacuum leaving a mixture of DEG and catalyst. The hexane was removed under vacuum distillation at 60° C. leaving the product alkoxylate and olefin. The olefin was then separated from the alkoxylate product via applying more heat to the mixture, leaving the alkoxylate product and, in some cases (70, 72, 74, and 75), olefin in the flask.

TABLE 1

| | | | | | | Calculated | | Olefins | | | |
|---|---|---|---|---|---|---|---|---|---|---|---|
| | | | | | | Yield | Alkoxylates | in | Final | Alkoxylates | Olefins |
| | | | | | Na | after | in coupled | coupled | Product | in final | in final |
| | | Alkyl | | | Citrate | coupling | product | product | Yield | product | product |
| LR# | Glycol | Br | Catalyst | Time | (g) | (wt %)¹ | (mole %)³ | (mole %)⁴ | (wt. %)^{2,6} | (mole %)^{6,7} | (mole %)^{6,7} |
| 26135-68 | DEG | 2-Br—C8 | CuBr2 | 60 min | 11 | 96 | 80.0 | 20.0 | 54 | 99 | 1 |
| 26135-69 | DEG | 2-Br—C8 | ZnBr2 | 60 min | 11 | 98 | 78.4 | 21.6 | 62 | 99 | 1 |
| 26135-70 | DEG | 2-Br—C8 | FeBr3 | 60 min | 11 | 96 | 67.9 | 32.1 | 56 | 93 | 7 |
| 26135-71 | DEG | 2-Br—C8 | CuBr2 | 10 min | 11 | 95 | 79.6 | 20.4 | 58 | 99 | 1 |
| 26135-71A | DEG | 2-Br—C8 | CuBr2 | 30 min | 11 | 11 | — | — | 58 | 99 | 1 |
| 26135-72 | DEG | 2-Br—C8 | CuBr2 | 10 min | 0 | 11 | — | — | 46 | 85 | 15 |
| 26135-73 | DEG | 2-Br—C8 | ZnBr2 | 10 min | 11 | 11 | — | — | 63 | 98 | 2 |
| 26135-74 | DEG | 2-Br—C8 | ZnBr2 | 10 min | 0 | 11 | — | — | 47 | 92 | 8 |
| 26135-75 | DEG/ | 2-Br—C8 | ZnBr2 | 10 min | 0 | 11 | — | — | 46 | 89 | 11 |
| | TEG⁵ | | | | | | | | | | |
| 26222-75A⁸ | TEG | 2-Br—C8 | ZnBr2 | 10 min | 0 | 96 | 90.6 | 9.4 | 93 | 99 | 1 |
| 26135-76 | DEG | 1-Br—C8 | ZnBr2 | 10 min | 0 | 99 | 79.2 | 20.8 | 110 | — | — |
| 26135-77 | DEG | 1-Cl—C8 | ZnBr2 | 60 min | 0 | 80⁹ | 80.6 | 19.4 | 68 | 99 | 1 |
| 26135-78 | DEG | 2,3-diBr—C8 | ZnBr2 | 10 min | 0 | 0¹⁰ | — | — | — | — | — |

¹weight percent (wt. %) ethoxylate + olefin products of coupling reaction basis the alkane feed calculated from a GC spectrum of the DAC reaction mixture
²wt. % alkoxylate + olefin products of step e) basis the alkane feed; measured by actual weighing of the final product
³mole % alkoxylates obtained from step e); measured from an uncalibrated GC trace of the DAC reaction mixture
⁴mole % olefins obtained from step e); measured from an uncalibrated GC trace of the DAC reaction mixture
⁵70-30 mole % DEG/TEG produced a 70-30 mole % mixture of DEG and TEG alkoxylates
⁶product was lost in the final separation steps due to inefficiencies in the small equipment used
⁷measured by NMR; 72, 74 and 75 had more olefin because the last distillation was incomplete
⁸extra extractions were used to capture as much of the product as possible; measured by actual weighing of the final product
⁹the DAC reaction was not complete in 60 min.
¹⁰no coupling recorded; the data is invalid because the testing method did not allow for a high enough temperature for di-substituted paraffin material to distill off the column and thus it did not pass through the detector
¹¹the GC broke down before all of the experiments were completed

The results of these experiments show that high conversion and selectivity of alkanes and/or alkyl halides to alkoxylates can be obtained using the process of this invention. The experiments show that copper bromide, zinc bromide, and iron bromide all can be used in the homogeneous catalyst system. Experiment 75 shows that a predetermined mixture of products can be obtained by choosing a mixture of nucleophilic materials for the coupling reaction. A 70-30 mole % mixture of DEG/TEG was used and the alkoxylate product had a 70-30 mole % mixture of the alkoxylates of DEG and TEG, respectively. Experiment 77 shows that the coupling reaction works when chlorine is used in place of bromine but the reaction rate is much slower. Na citrate is used in several of the examples to remove HBr—this reaction takes place very quickly. Comparing the results of experiments 71 & 72 and 73 & 74, it can be seen that, contrary to expectations that most of the product would be lost if HBr was not removed right away, only 20% or so of the product was lost when the HBr was retained. This means that the step of removing HBr with Na citrate or some other such material can be eliminated and a technique such as inert gas (for instance, nitrogen) sparging can be used effectively to remove the HBr. Experiment 75 was repeated in 75A with additional extractions to capture as much of the product as possible. This experiment shows that 93% yield can be obtained. This yield compares very favorably to the 96% yield calculated from the products in the coupling reaction mixture.

1. A process which comprises reacting alkyl halides with a nucleophilic material in the presence of a homogeneous catalyst system to produce derivatives of alkyl halides, wherein the homogeneous catalyst system comprises at least one metal or metal compound which has the ability to form metal-halogen bonds.

2. The process of claim 1 wherein the halogen is bromine.

3. The process of claim 1 wherein the nucleophilic material contains oxygen, nitrogen, or sulfur.

4. The process of claim 1 wherein the nucleophilic material is selected from the group consisting of polyethylene glycols, polypropylene glycols, diethylene glycol, triethylene glycol, dimethylaminopropylamine, CH₃SH, monopropylene glycol and monoethylene glycol.

5. The process of claim 1 wherein the metal in the homogeneous catalyst system is selected from the group consisting of Groups VIII, IB, and IIB of the periodic table of the elements, CAS version.

6. The process of claim 5 wherein the metal in the homogeneous catalyst system is zinc.

7. The process of claim 1 wherein specific mixtures of reaction products are produced by selecting a desired mixture of nucleophilic materials.

8. The process of claim 1 wherein the product is an alkyl alcohol alkoxylate.

9. A process which comprises halogenating paraffins to form alkyl halides and reacting the alkyl halides with a nucleophilic material in the presence of a homogeneous catalyst system to produce derivatives of alkyl halides, wherein the homogeneous catalyst system comprises at least one metal or metal compound which has the ability to form metal-halogen bonds.

10. The process of claim 9 wherein the halogen is bromine.

11. The process of claim 9 or wherein the nucleophilic material contains oxygen, nitrogen, or sulfur.

12. The process of claim 9 to wherein the nucleophilic material is selected from the group consisting of polyethylene glycols, polypropylene glycols, diethylene glycol, triethylene glycol, dimethylaminopropylamine, CH₃SH, monopropylene glycol and monoethylene glycol.

13. The process of claim 9 wherein the metal in the homogeneous catalyst system is selected from the group consisting of Groups VIII, IB, and IIB of the periodic table of the elements, CAS version.

14. The process of claim 13 wherein the metal in the homogeneous catalyst system is zinc.

15. The process of claim 9 wherein specific mixtures of reaction products are produced by selecting a desired mixture of nucleophilic materials.

16. The process of claim 9 wherein the product is an alkyl alcohol alkoxylate.

17. A method for enhanced oil recovery which comprises:
a) reacting alkyl halides with a nucleophilic material in the presence of a homogeneous catalyst system to produce derivatives of alkyl halides, wherein the homogeneous catalyst system comprises at least one metal or metal compound which has the ability to form metal-halogen bonds;
b) providing the derivatives to at least a portion of a hydrocarbon containing formation; and
c) allowing the derivatives to interact with hydrocarbons in the hydrocarbon containing formation.

18. (canceled)

19. (canceled)

20. (canceled)

21. (canceled)

22. (canceled)

23. A method as claimed in claim 17 further comprising halogenating paraffins to form alkyl halides for reacting with the nucleophilic material.
